(19) United States
(12) Patent Application Publication
(10) Pub. No.: US 2026/0069242 A1
(43) Pub. Date: Mar. 12, 2026

(54) WIRELESS ICE CATHETER SYSTEMS

(71) Applicant: yoR Labs, Inc., Beaverton, OR (US)

(72) Inventors: Glen Wallace Mclaughlin, Woodside, CA (US); Anshumali Roy, Portland, OR (US); Dakota Pellegrino, Hillsboro, OR (US); Andrei Nikolaevich Kniazev, Happy Valley, OR (US)

(21) Appl. No.: 19/109,062

(22) PCT Filed: Sep. 7, 2023
(86) PCT No.: PCT/US2023/073684

§ 371 Date: Mar. 5, 2025

Related U.S. Application Data
(60) Provisional application No. 63/375,103, filed on Sep. 9, 2022.

(51) Int. Cl. A61B 8/12; A61B 8/00; A61B 8/08

(52) U.S. Cl. CPC A61B 8/12; A61B 8/0883; A61B 8/4472; A61B 8/4488; A61B 8/461; A61B 8/54; A61B 8/56

(57) ABSTRACT

An intracardiac echocardiography (ICE) catheter system that includes a control system disposed in a portable housing, the control system including a battery, and an ICE ultrasound circuit connected to the battery. The ICE ultrasound circuit is configured to be connected to be in communication with an ICE catheter and process data received from an ultrasonic array positioned of the ICE catheter. The Ice catheter system also includes a computing module having electronics and software loaded thereon capable of processing data, and a wireless module connected to the battery and the computing module, the wireless module configured to wirelessly communicate signals to a control counsel in one or more displays that are configured for use in an ICE catheter procedure. A catheter handle can be configured to include the control system obviating the need for a cable connecting the catheter handle in the control system.

### INCORPORATION BY REFERENCE TO ANY PRIORITY APPLICATIONS

Any and all application for which a foreign or domestic priority claim is identified in the Application Data Sheet as filed with the present application are hereby incorporated by reference under 37 CFR 1.57. This disclosure is related to PCT Application PCT/US2023/073684, filed Sep. 7, 2023, and U.S. Provisional No. 63/375,103, filed on Sep. 9, 2022, both of which are incorporated by reference herein in their entirety.

### TECHNICAL FIELD

The invention relates to catheter medical systems. Specifically, the invention relates to catheter devices for cardiac or vascular diagnostic and interventional procedures.

### BACKGROUND

Intracardiac echocardiography (ICE) catheter systems, including all of the associated equipment for displaying a processing ICE images, can be implemented in multiple large equipment housings connected with a plurality of cables. Often ICE catheter systems are used in a hospital room (e.g., a catheterization lab or “cath lab”) having limited space and in particular limited floorspace. Consequently, the multiple large pieces of equipment and cables can occupy much of the available space in the little room for medical practitioners and other necessary equipment. It would be advantageous to minimize the amount of equipment and cables, and the size of the equipment, needed for an ICE procedure.

### SUMMARY

Certain aspects of this invention are defined by the independent claims. The dependent claims concern optional features of some embodiments of the invention. The systems, methods, and devices described herein each have several aspects, no single one of which is solely responsible for its desirable attributes. Without limiting the scope of this disclosure, several non-limiting features will now be discussed briefly.

Embodiments relate to an integrated wireless ultrasound system for intracardiac echocardiography (ICE). One innovation includes an ICE catheter system that includes an ICE catheter control system (“control system”) in a small portable housing that includes its own power source. The ICE catheter system can also include a catheter handle, and an ICE catheter which is removably couplable to the catheter handle. The ICE catheter will may be provided separate from the catheter handle and coupled to the catheter handle prior to its use for an ICE procedure. The catheter is typically disposable. In some embodiments, the catheter handle is a durable/reusable handle. In some embodiments, the catheter handle includes one or more controls (e.g., control buttons or switches) that can control a function of collecting intracardiac echocardiography images. In some embodiments, the catheter handle includes a base portion that is structured to hold the catheter handle at a certain orientation when the base portion is placed on a surface, for example, on a leg or another portion of a patient, or on a table, bench or other piece of equipment.

In some embodiments, when in use the catheter handle (“handle) is coupled to the ice catheter control system by a communication cable (or “link cable”). The link cable includes a distal portion that is configured to couple to the handle and a proximal portion that is configured to be connected to the control system. The coupling of the link cable to the handle can include a mechanical connection and electrical connections. In some embodiments, the catheter handle can be configured with an aperture to receive a distal portion of the link cable and mechanically and electrically couple the link cable to the handle. When the link cable is coupled to the handle, the control buttons of the catheter handle are electrically connected to the link cable, and correspondingly the control system. When the link cable is coupled to the handle (e.g., on a proximal end of the catheter handle), and an ICE catheter is also coupled to the handle (e. g, on a distal end of the handle), a portion of the link cable is electrically coupled to the ICE catheter, either directly or through an electrical interface of the handle.

Embodiments of the control system include a battery module comprising one or more batteries or power sources, and electrical port configured to connect with the link cable. The link cable provides a communication channel for communicating echocardiography signals from the ICE catheter/handle assembly to the control system, and for communicating control signals from the control system to the ICE catheter. In some embodiments, the control system is configured to communicate signals to the handle, and receive control signals from the handle (e.g., from one or more controls of the handle).

The control system can also include a wireless module configured for communicating wirelessly with peripheral equipment, obviating the need to have additional hardware cables connecting a variety of equipment. The peripheral equipment can include, for example, the control console, one or more displays, and other computer equipment. The control system can further include specific ICE ultrasound circuitry for receiving and processing ultrasound data and controlling an ultrasound array of the ICE catheter, a compute module having electronics and software residing thereon capable of further processing image and data acquired from the ICE catheter, provide control functionality, and communication functionality in any additional functionality that is needed when performing an intracardiac echocardiography procedure.

In other embodiments, the control system can be fully integrated a durable catheter handle. In such embodiments, an ICE catheter is coupled to the catheter handle and is controlled by the control system in the catheter handle. Such a control system also includes ICE ultrasound circuitry, the compute module, a wireless module, and battery pack, as described above. In this embodiment, the catheter handle can communicate wirelessly with peripheral equipment, peripheral equipment including, for example, a control console, one or more displays, and other computer equipment. Having the control system fully integrated into the durable catheter handle further reduces the number cables of the system and that have to be dealt with while performing an ICE catheter procedure.

In one aspect, an intracardiac echocardiography (ICE) catheter system includes a control system disposed in a portable housing, the control system including a battery; an ICE ultrasound circuit connected to the battery, the ICE ultrasound circuit configured to be connected to an ICE catheter and process data received from an ultrasonic array positioned of the ICE catheter; a computing module having electronics and software loaded thereon capable of processing data; and a wireless module connected to the battery and the computing module, the wireless module configured to wirelessly communicate signals to a control counsel in one or more displays that are configured for use in an ICE catheter procedure. In some aspects, the ICE catheter system further includes a durable catheter handle, and a link cable, the link cable having a distal end configured to be connected to the catheter handle and the proximal end configured to be connected to the control system, the link cable configured to communicate signals between the catheter handle and the control system and communicate signals between the catheter connected to the catheter handle in the control system. The control system is configured to operate the catheter handle and the ICE catheter using power from the battery without another energy source. The computing module is configured to determine a processing capability of the control system and determine a processing capability of the control console; and determine operations related to processing ICE images to be performed in the control system and operations related to processing ICE images to be performed in the control console based on the determined processing capabilities of the control system and the control counsel. The computing module is further configured to communicate, via the wireless module, information for processing ICE images in the control console, to the control console.

In one aspect, an intracardiac echocardiography (ICE) catheter system, includes a catheter handle including a control system disposed in the catheter handle, the control system including a battery; an ICE ultrasound circuit connected to the battery, the ICE ultrasound circuit configured to be connected to an ICE catheter and process data received from an ultrasonic array positioned of the ICE catheter; a computing module having electronics and software loaded thereon capable of processing data; and a wireless module connected to the battery and the computing module, the wireless module configured to wirelessly communicate signals to a control counsel in one or more displays that are configured for use in an ICE catheter procedure. The catheter handle is configured to a reusable handle. The control system is configured to operate the catheter handle and the ICE catheter using power from the battery without another energy source. The computing module is configured to determine a processing capability of the control system and determine a processing capability of a control console; and determine operations related to processing ICE images to be performed in the control system and operations related to processing ICE images to be performed in the control console based on the determined processing capabilities of the control system and the control counsel. The computing module is further configured to communicate, via the wireless module, information for processing ICE images to the control console.

In one aspect, a method of processing intracardiac echocardiography (ICE) catheter data includes: determining a processing capability of a plurality of units being used in an ICE session, each of the units having at least one computer hardware processor configured to process ICE data, where a first one of the units receives ICE data during an ICE procedure and the first one of the units is in communication with the other of the plurality of units; providing ICE data from the first one of the units to at least one other of the plurality of units; processing the ICE data on the first one of the plurality of units and the at least one other of the plurality of units; and aggregating processed ICE data from each of the first one of the plurality of units and the at least one other of the plurality of units; and displaying the aggregated processed ICE data as an image on a display. The processing of the ICE data on the first one of the plurality of units and the at least one other of the plurality of units occurs simultaneously. One of the plurality of units is an ICE unit. One of the plurality of units is an ICE catheter handle unit. One of the plurality of units is a console unit. One of the plurality of units is a remote unit. Providing ICE data from the first one of the units to at least one other of the plurality of units comprises providing ICE data from the first one of the units to at least one other of the plurality of units via a wireless communication channel.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a schematic illustrating an example embodiment of a wireless ICE system.

FIG. 2 is a schematic illustrating another example embodiment of a wireless ICE system, where a control system of the wireless ICE catheter system is incorporated in a catheter handle (e.g., a durable/reusable catheter handle).

FIG. 3 is a perspective view of the distal end of a catheter handle (e.g., a durable catheter handle) that can be used in a wireless ICE catheter system.

FIG. 4 is a perspective view of the proximal end of a catheter handle (e.g., a durable catheter handle) that can be used in a wireless ICE catheter system.

FIG. 5 illustrates an example of a wireless ICE system with distributed controls and computing, according to a first embodiment.

FIG. 6 illustrates an example of a wireless ICE system with distributed controls and computing, according to a second embodiment.

FIG. 7 illustrates an example of a wireless ICE system with distributed controls and computing, according to a third embodiment.

FIG. 8 illustrates fourth example of a wireless ICE system with distributed controls and computing, according to a first embodiment.

FIG. 9 illustrates an example of a wireless ICE system with distributed controls and computing, according to a fifth embodiment.

FIG. 10 illustrates an example of multi-mode imaging control structure across a wireless ICE system, according to some embodiments.

FIG. 11 illustrates an example of multi-mode load balancing across a wireless ICE system, according to some embodiments.

### DETAILED DESCRIPTION OF CERTAIN INVENTIVE ASPECTS

Ever since the invention of the modern disposable catheter in 1940s, it has enjoyed rapid growth and wide expansion in the medical fields. To date, catheters have found applications in treating cardiovascular, urological, gastrointestinal, neurovascular, and ophthalmic diseases. Especially in the area of treating cardiovascular diseases there exist many catheter modalities. Among them are intracardiac echocardiography (ICE), intravascular ultrasound (IVUS), radiofrequency (RF) ablation, and fractional flow reserve (FFR). Intracardiac echocardiography (ICE) adopts a microscopic ultrasound array to realize direct imaging of anatomical cardiac structures in heart. It greatly helps procedures such as radiofrequency (RF) ablation. Intravascular ultrasound (IVUS), on the other hand, uses a microscopic ultrasonic array to generate images of a blood vessel, i.e., coronary artery. Radiofrequency (RF) ablation applies heat to destroy diseased tissue in order to stop pain. And fractional flow reserve (FFR) adopts a pressure sensor to measure pressure difference across a coronary artery stenosis to determine its effect on oxygen delivery to the heart tissue.

A catheter modality device normally comprises two parts, a hardware system having electronics with software build-in to send control signals to manipulate the procedure and to acquire and process data for display and operation, and a disposable catheter which usually includes a catheter tip, a handle to operate the catheter, and an electrical connector to connect to the hardware system. Generally, the hardware system is disposed on a wheeled platform so that the device can be easily moved to different places, for example, a lab, a procedure room, or a storage room. Different modalities are developed to treat different diseases. And even a single modality can have different devices developed by different companies potentially adopting different technologies for signal processing and data computing. The result is that each device has its own hardware system and catheter, and no two catheter devices share common hardware. Therefore, a hospital normally needs a large storage room to house and maintain all types of medical equipment, including many catheter devices, and a lab or procedure room needs to be large enough to hold at least few devices. In many hospitals, and in particular underdeveloped countries and areas, hospitals are usually tight of space and include numerous cables that may be on the floor or hung across the room, which may obstruct and/or hinder performing medical procedures and can be a trip hazard, and thus it would be advantageous to minimize number the number of cables used in a catheterization lab, hospital room, or other medical facilities.

An ICE catheter system that is small in size for better portability, and that it connects wirelessly to control console, display or displays and other peripheral equipment would be advantageous. Such an ICE catheter system can be moved/transported easily and when in-use occupies a minimum amount of space and requires few, if any, cables. The systems disclosed herein relate to a small high performance ICE ultrasound system. In an example, the ICE catheter system is a small brick-size ultrasound system with its single/primary modality being intracardiac echocardiography. Generally, in an embodiment where the control system is separate from the catheter handle, the control system can be disposed within a housing that is about, for example, 12″×10″×4″. This system can include an internal data acquisition and processing circuitry that is specific to ICE in terms of channel count, electronic circuitry design and tuning, and onboard computation module(s) with software layers to implement ICE ultrasound image reconstruction. It will utilize a variety of other software application layers for data, control and metrology information between itself and a control console and to an electronic picture archiving and communication system (PACS)/electronic medical record (EMR) system.

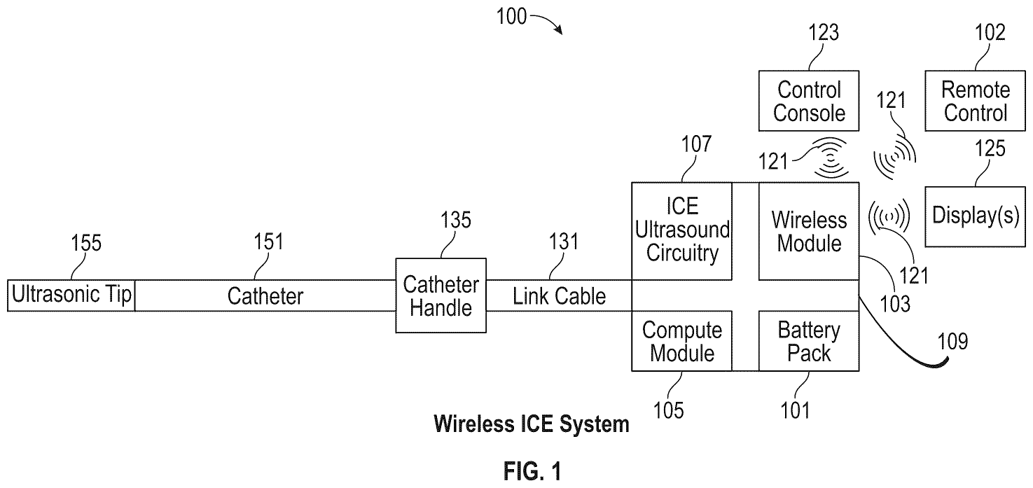

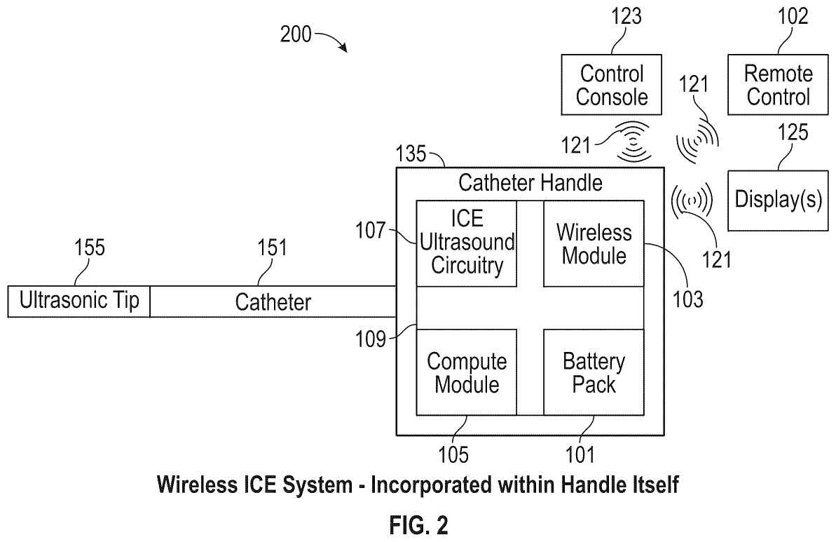

In an embodiment, the catheter system is fully wireless. The system can be wireless in terms of both: (1) one or multiple wireless displays via internal wireless module and software layer to achieve such. and (2) battery powered system capable of running either off of ac mains, or standalone off of battery power. To accomplish this, It'll have rechargeable batteries sufficient for half a day of continuous operation (or full day of typical use). The system will utilize (possibly) custom connectors to connect to an ICE catheter, and/or a specific yoRLabs catheter. The control system can have a lower delivery in thermal dissipation systems adequate to the power needs of the system on supply side. A combination of active and passive cooling methods for thermal management. This would also involve multiple sensors for system monitoring tasks and control systems for management of power delivery and heat removal. In some embodiments, the control system will be contained in a housing (see FIG. 1) that can be coupled to the catheter handle via link cable, and the control system is configured to communicate wirelessly with other peripheral equipment, for example, one or more displays, control console, and the like. In some embodiments, the control system is fully contained in the catheter handle itself, as illustrated in FIG. 2. In this embodiment, the control system (in the catheter handle) is configured to communicate wirelessly with other peripheral equipment, for example, one or more displays, control console, and the like.

### List of Certain Components

The following is a list of certain components that are described and enumerated in this disclosure in reference to the above-listed figures. However, any aspect of the devices illustrated in the figures, whether or not named out separately herein, can form a portion of various embodiments of the invention and may provide basis for claim limitation relating to such aspects, with or without additional description. Generally, herein, reference to a “distal” portion indicates a portion/component that is positioned farthest from the medical practitioner and closest to the patient when the device/component is in use (e.g., during an intracardiac echocardiography procedure), and reference to a “proximal” portion indicates a portion/component that is positioned closer to the medical practitioner using the system farther from the patient when the device/component is in use. The enumerated components include:
100 embodiment of wireless ICE system
101 battery pack
102 remote control
103 wireless module
105 computing module
107 ICE ultrasound circuitry module
109 control system
110 proximal end, handle
112 distal end, handle
121 wireless signal
123 control console
125 display(s)
131 link cable
135 catheter handle
137 body
139 tail (rotatable)
141 catheter steering controls
143 base/support feet
145 pushrods
147 control buttons
148 internal space/aperture for receiving distal end of link cable
149 contact pads
151 catheter
155 catheter tip
137 cylindrical handle shell
200 embodiment of wireless ICE system in a catheter handle

### Illustrative Examples of a Wireless ICE Catheter System

FIG. 1 is a schematic illustrating an example embodiment of a wireless ICE system. This embodiment of an ICE catheter system 100 includes a control system 109 that is configured to be coupled to a catheter handle 135 via a link cable 131, and communicate with a control console 123, one or more displays 125, and a remote control 102. The control system 109 can include modules made of PCBs and electronic components. In this example, control system 109 includes a battery pack 101, a wireless module 103, a computing module 105, and an ICE circuitry module 107.

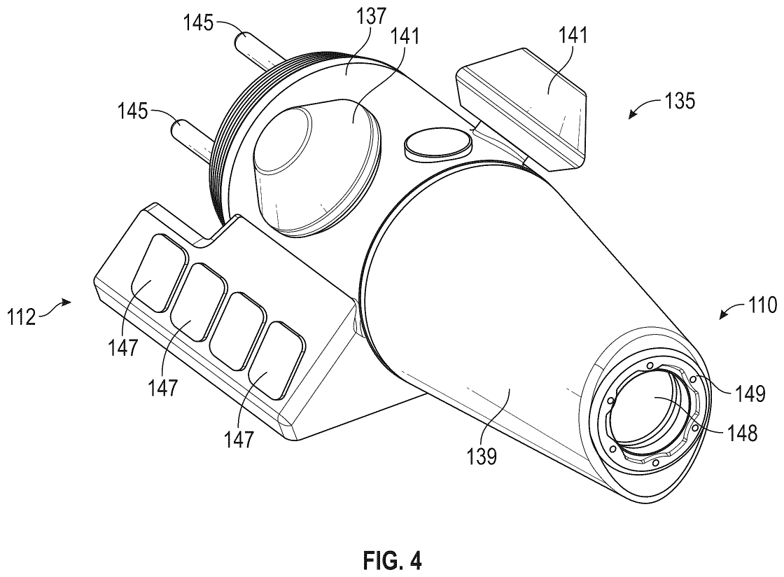

The control system 109 communicates with the catheter 151 in the catheter handle 135 via the link cable 131. The distal end of the link cable 131 is configured to electrically and mechanically coupled to the catheter handle 135. A proximal end of the link cable 131 is configured to electrically couple to the control system 109. In some examples, the proximal end of the link cable 131 includes a male electrical connector in the control system 109 includes a corresponding female connector which the proximal end of the link cable 131 can be connected to. The link cable 131 can be electrically connected to the catheter handle 135 via one or more contacts positioned around an opening on the proximal end of the handle 135 (See FIG. 4). The link cable 131 can also be electrically connected to the catheter 151, and the ultrasonic array 155 at the tip of the catheter 151, via the electrical interface at the distal end of the link cable 131.

The control system 109 includes modules and components assembled in a small chassis for easy storage and transportation. The battery pack 101, preferably disposed in a compartment in the chassis, supplies power to run the hardware system, the catheter 151 and the catheter handle 135. Preferably, the battery pack 101 consists of rechargeable battery set and has a battery charger disposed in the chassis. In this way the control system 109 can be powered by AC from wall outlet or powered by the battery pack 101 when standalone. When fully charged and running standalone, it is preferred that the battery pack 101 can support the multiple-modality system 100 to operate for at least 4 hours, and more preferably more than 6 hours. The battery pack 101 can also adopt non-rechargeable batteries. In this case, the multiple-modality system 100 preferably has built-in AC power connection.

The wireless module 103 have electronics and software loaded thereon to connect the multiple-modality system 100 with a display or displays 125, a control console 123, which may be a laptop computer or a tablet computer, and potentially other peripheral accessories, through wireless signal 121. The wireless communication is preferably based on a short-range wireless protocol, such as Bluetooth®, WiFi, or ZigBee®.

The computing module 105 includes microelectronics, such as ASIC, CPU, GPU or FPGA, other electrical components and software loaded on a memory chip or a hard drive to process image data and to send control commands to the catheter 151 or handle 135. The modality circuitry module 107, on the other hand, funnels data between the catheter 151 and the computing module 105. When it receives data from the catheter 151, it may carry out some basic processing, i.e., data conversion, before sending the data to the computing module 105. The computing module 105 then performs tasks such as graphics processing and data analysis and sends the processed data to the control console 123 and the display(s) 125. Upon reviewing the displayed graphics and the data results, the physician who operates the control system 109 makes decision to send out commands from the control console 123 to perform certain functional actions at the catheter tip 155 or to perform certain further data analysis.

The modules of the control system 109 shown in FIG. 1 are preferably assembled in a housing or chassis that is sized within 15 inch×12 inch×6 inch, and preferably within 12 inch×10 inch×4 inch. Such a multiple-modality system in a small chassis can be carried around easily. It can be stored on a shelf or in a cabinet in a storage room, taking only a small place. And it can easily be installed in a lab or procedure room even for those hospitals with tight space. If needed, it can be stacked with other equipment. Furthermore, the wireless connection capability allows the control system 109 to share peripheral equipment, such as control console 123 and display(s) 125 with other devices in the room. As such, moving the control system 109 is usually not accompanied by moving peripheral equipment.

The modules of the control system 109, including the battery pack 101, the wireless module 103, the computing module 105 and the modality circuitry module 107, can be built into a plurality of PCBs and electronic components assembled to the chassis, with connector(s) on a chassis wall to connect to the link cable 131. In other embodiments, the electronics of the computing module 105 and the wireless module 103 can be mounted on a mother PCB that is installed in the chassis, and the modality circuitry module 107 is then connected to the mother PCB as a child PCB. In this case, the modality circuitry module 107 child PCB may have a connector, or two connectors built on it. When assembled, the connector(s) on the modality circuitry module 107 child PCB is exposed through a wall on the chassis to connect to the link cable 131.

For optimal system performance, a combination of active and passive cooling methods is employed for thermal management. This may involve multiple sensors installed in the chassis of the control system 109 to monitor temperature to activate power delivery management and heat removal.

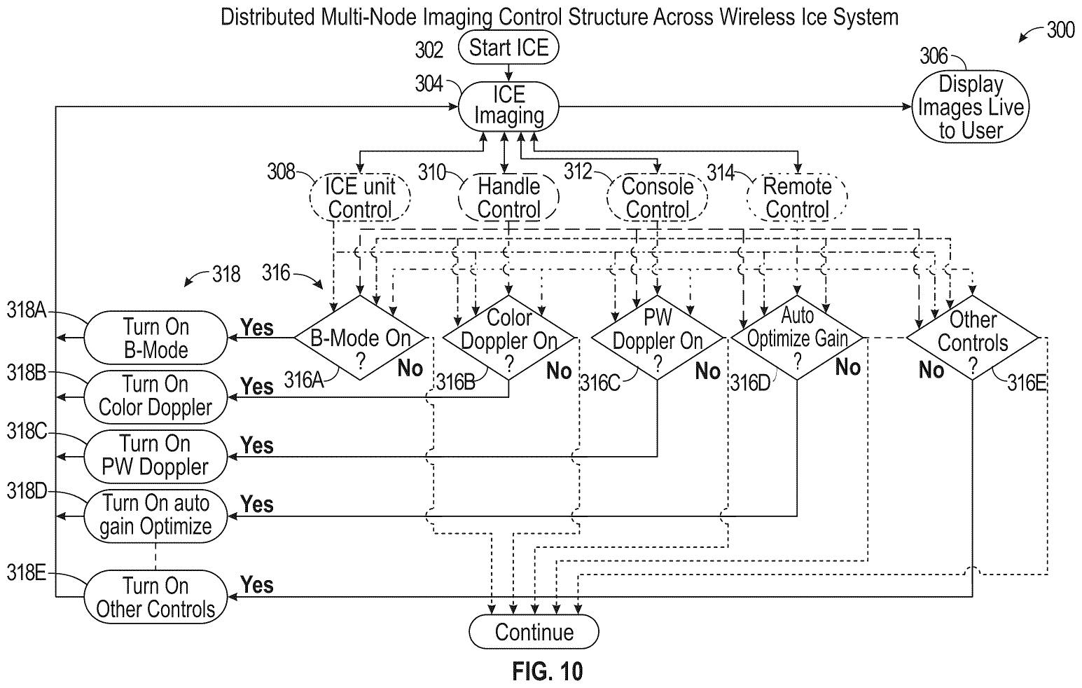

The ICE ultrasound circuitry module 107 shown in FIG. 1 supports intracardiac echocardiography (ICE). In some embodiments, a circuitry module can be configured to support other types of catheters, e.g., intravascular ultrasound (IVUS). As discussed above, the modality circuitry module 107 may be built as a child PCB adapted to be connected to the mother PCB that includes the computing module 105 and the wireless module 123. The remote control 102 can be used to remotely control one or more functions or processes during an ICE session, as described further in reference to FIG. 10.

FIG. 2 is a schematic illustrating another example embodiment of a wireless ICE system 200. Many aspects of the functionality of the wireless ICE system 200 is similar, or the same, as the system shown in FIG. 1. However, in the embodiment illustrated in FIG. 2, the control system 109 is incorporated in the catheter handle 135 (e.g., a durable/reusable catheter handle). The control system 109 includes ICE ultrasound circuitry 107, a computing module 105, a wireless module 103, and a battery pack 101, and these components can operate the same way as described embodiment of FIG. 1. In this embodiment, because the control system 109 corporative in the catheter handle itself, there is no need for a link cable connecting the catheter handle to the control system. Also, because the catheter handle 135 now includes a wireless module 103, the catheter handle 135 itself can communicate wirelessly 121 with other peripheral equipment, for example, a control console 123, one or more displays 125, remote control 102, and other related equipment that may be used in an ICE procedure.

As progress has been made in the semiconductor industry, component density on IC chips and PCBs has been increasing. It is possible to build each module on a high-density PCB, an IC chip, or even a chiplet. When all the components are assembled, the assembly is small enough to fit into the internal space provided by the catheter handle. For example, each of the compute module 105, wireless module 103 and ICE ultrasound circuitry 107 can be designed and built as an IC chip. And the IC chips can be mounted on a high-density PCB to fit into the handle. Or the functionalities of the modules can be distributed to a plurality of IC chips mounted on one or a few high-density PCBs small enough to fit inside the catheter handle. The battery pack 101 is preferably a rechargeable cell battery potentially integrated with a charger. The control system 109 in a catheter handle communicates wirelessly with the control console 123 and the display(s) 125, similar to the system 100 in FIG. 1.

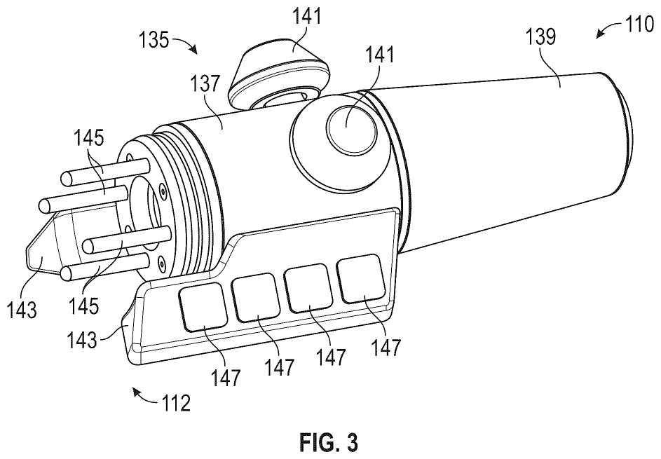

FIG. 3 is a perspective view of the distal end of a catheter handle 135 (e.g., a durable catheter handle) that can be used in a wireless ICE catheter system, for example, the system illustrated in FIG. 1. FIG. 4 is a perspective view of the proximal end of the catheter handle shown in FIG. 3. As illustrated in FIGS. 3 and 4, the catheter handle 135 can include a body portion (“body”) 137 having a cylindrical shell housing and generally positioned on the distal end of the handle 135, and a tail portion (“tail”) 139 generally positioned on the proximal end of the handle 135. The tail 139 is connected to coupling structure for attaching a catheter on the distal end 112 of the handle, for example, pushrods 145. The tail 139 configured to be rotatable relative to the body 137, and when the tail 139 is rotated, the coupling structure and a catheter attached to the distal end 112 of the handle 135 also rotates. In this embodiment, the handle 135 also includes a base 143 having to support feet, the base 1443 configured to support the handle 135 when the handle is placed on a surface (e.g., a portion of a patient, the table, etc.).

In this embodiment, the handle also includes two control knobs 141 that can be rotated to operate the connected catheter. By rotating the control knobs 141, the pushrods 145 are actuated to tilt a swashplate (not shown) in contact with the pushrods, so as to bend and steer the catheter tip 155. Control buttons 147 located on the base 143 may be used to control various functions of the catheter, including imaging functions. In other embodiments, control buttons can be positioned on other portions of the handle, or other portions of the base.

An internal space 148 of the inner cylindrical body 139 is configured to hold the distal end of the link cable 131 that is connected to the control system at the proximal end (e.g., for the embodiment of FIG. 1 As the distal end of the link cable 131 is inserted into the internal space 148, it has features to be locked in place while electrical connections are made to connect the link cable 139 to the contact pads 149 at the proximal end of the inner cylindrical body 139. Meanwhile, the distal end of the link cable 131 is electrically connected to an electrical interface which is adapted to connect with a connector at the proximal end of the catheter 151 to realize an electrical connection between the link cable 131 and the catheter 151. In other embodiments, the distal end of the link cable 131 has electrical connectors to directly connect to the proximal end of the catheter 151. As such the adaptor mechanism in the handle 135 is not needed. The distal end of the link cable 131 can be keyed in the inner cylindrical body 139 by cross-sectional shape feature, so that rotating the inner cylindrical body 139 will bring the distal end of the link cable 131 and the catheter 151 to rotate together.

The universal catheter handle 135 constructed as described above can be a reusable catheter handle. When the link cable 131 is unplugged from the handle 135 and the catheter 151 is removed, the catheter handle 135 can be sterilized. Then it can be used in the next procedure.

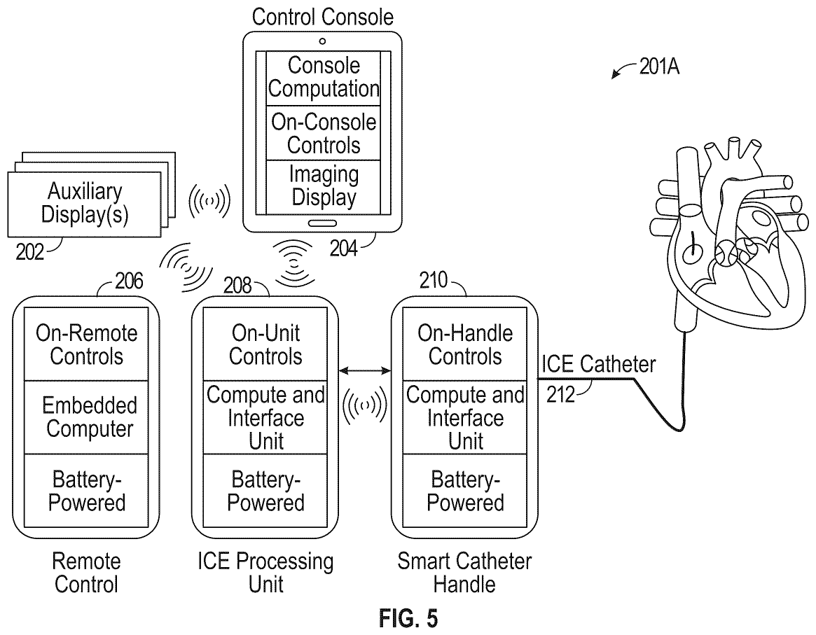

FIGS. 5-9 illustrate an examples of wireless ICE systems with distributed controls and computing. In such configurations, the communication between one or more control console displays and power will have the ability to be wireless. This can be implemented various ways. For example, in one embodiment of the system, the system includes a smart catheter handle, an ultrasound processing system, a console, and one or more displays that are configured to communicate wirelessly. In another embodiment, the system includes a smart catheter handle, a console, and one or more other wireless displays. Some embodiments can use, for example, a catheter handle similar to the design of the catheter handle illustrated in FIGS. 3 and 4.

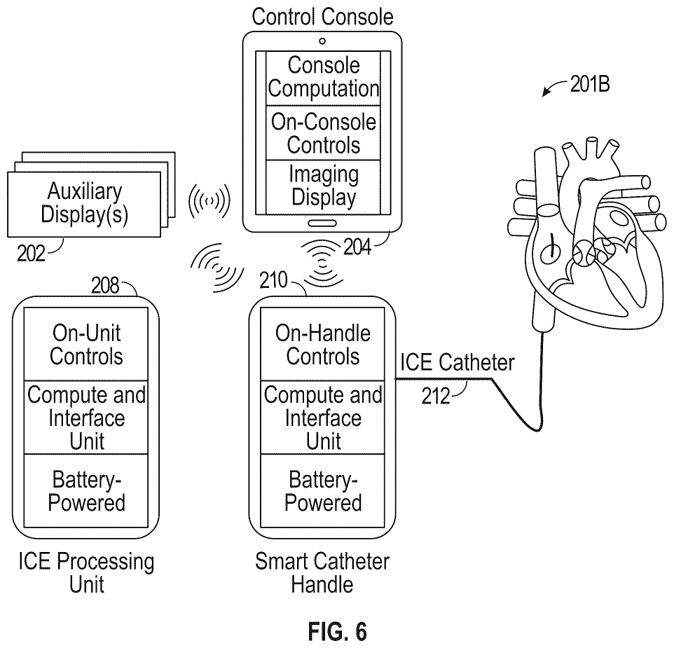

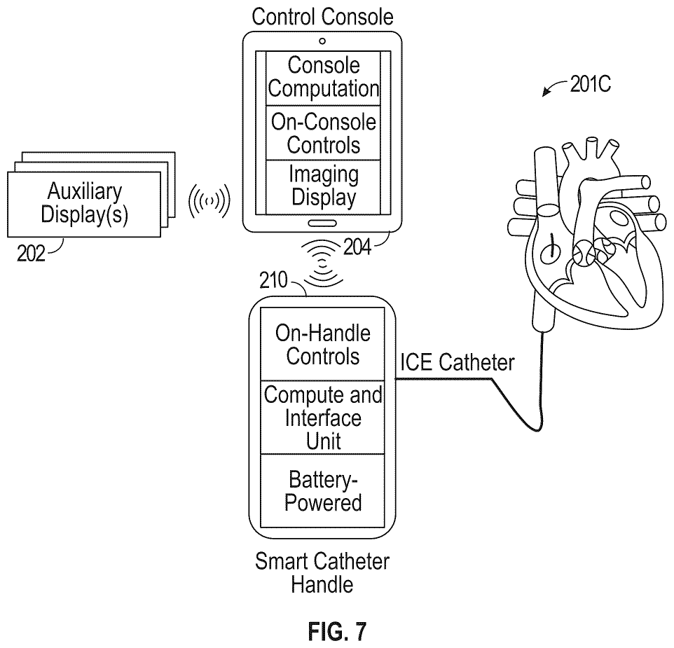

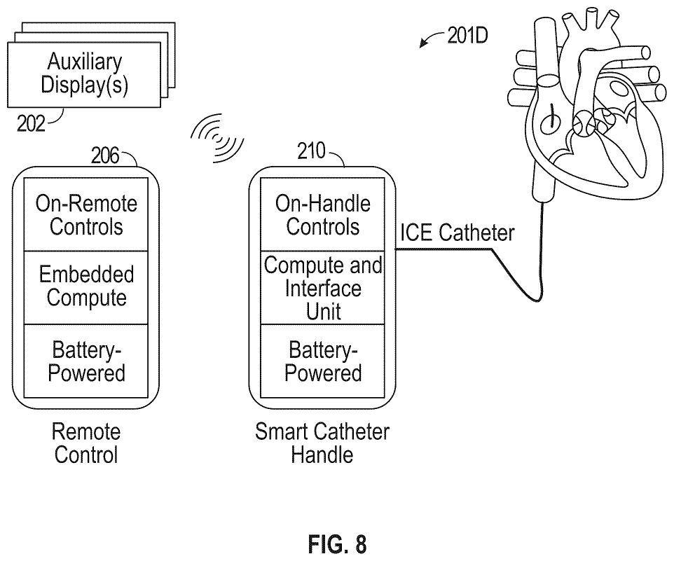

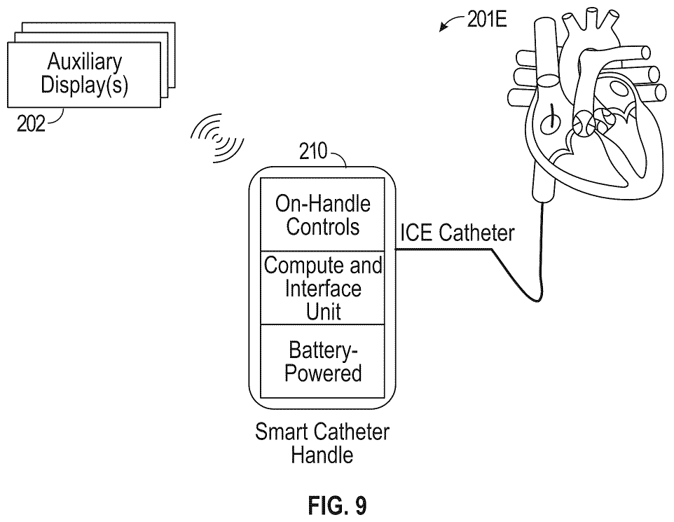

FIG. 5 illustrates an example of a wireless ICE system with distributed controls and computing, according to a first embodiment. Various other embodiments are contemplated. For example, FIG. 6 illustrates an example of a wireless ICE system with distributed controls and computing, according to a second embodiment. FIG. 7 illustrates an example of a wireless ICE system with distributed controls and computing, according to a third embodiment. FIG. 8 illustrates fourth example of a wireless ICE system with distributed controls and computing, according to a first embodiment. FIG. 9 illustrates an example of a wireless ICE system with distributed controls and computing, according to a fifth embodiment.

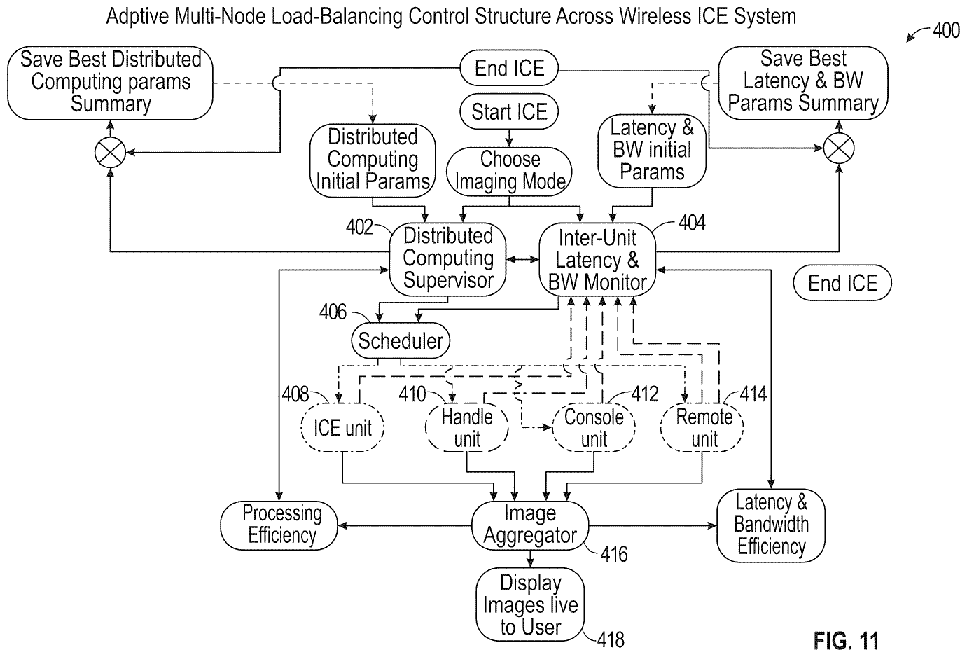

FIGS. 10 and 11 illustrate control structure across a wireless ICE system. For example, FIG. 10 illustrates an example of multi-mode imaging control structure across a wireless ICE system, according to some embodiments. FIG. 11 illustrates an example of multi-mode load balancing across a wireless ICE system, according to some embodiments.

Referring to FIG. 10, various embodiments are configured for full imaging control manipulation via configurable (programmable function) buttons across multiple nodes within the system. For example, controls for functions or processes that are used in ICE session may be implemented on (a) the handle itself, (b) the ICE unit, (c) the Console interface computer, and/or (d) the remote control unit. In various embodiments, any combination of one or more controls on the handle, the base unit, the console interface computer, and the remote control unit, are contemplated (i.e., all subset combinations are contemplated and can be implemented in various embodiments). In some embodiments, the controls can be actuated by voice commands. Multi-node imaging control structure are advantageous to be used during an ICE session because, for example, they can distribute control and responsibility of the various functions to the team of medical practitioners involved in the ICE session.

Referring to the process flow chart of FIG. 11, a Distributed Computing supervisor process constantly monitors available processing power on all of the available processing units in the system, and decides what the best computing load split would be such that each processing unit performs a determined amount of the processing required for the ICE process. The processing can include, for example, image processing of received ICE data to form and display generated images based on the received data, feature detection, and all other computing tasks performed to generate and analyze ICE data. The processing units can include, for example, but not limited to, an ICE processing unit, a smart catheter handle, a control console, displays, and the like, depending on the system implementation being used. As an example, the systems of FIGS. 5-9 illustrate different system implementations, and any of the components of the system that have computing capability can be used as a computing resource.

During processing of ICE data, an imaging mode can be selected. An Inter-unit Latency Monitor constantly monitors what the wireless latency and bandwidth between each of the units is. For example, what the wireless latency and the bandwidth is for each of the ICE unit, the handle unit, the Council unit, and the remote unit. The Distributed Computing Supervisor and the Inter-unit latency/bandwidth (BW) monitor then agree on what the final load split should be based on available computing power, bandwidth, and latency. This information is then passed on to a Scheduler process.

At this point the Scheduler launches various aspects of the imaging task to each of the units (e.g., the ICE unit, handle unit, console unit, and/or remote unit). Each of the sub-tasks may include directly working on RF (raw digitized) data or pixel-based post-processing tasks, or any other computing task needed during the ICE session. The Image Aggregator can receive information from each of the units processing the data, and composites the final image (and/o graphics, and/or other information) to be displayed and sends it to one or more of the display panels for display. Physically, the Image Aggregator can be resident on any one or more of the compute units.

At end of each cycle through the process, a summary of processing efficiency of last the task is sent back to the Distributed Computing Supervisor. Also, a summary of latency and bandwidth efficiency of last task is sent back to the Inter-unit latency and Bandwidth Monitor (that's why the bent arrow is a 2-way arrow).

As the imaging acquisition proceeds through, both the distributed computing supervisor and the inter-unit latency and BW monitor get more efficient at allocating resources for most efficient imaging. At end of an imaging session, the best combination for both distributed computing and for latency and bandwidth for each imaging modality used during the ICE session are saved. Every subsequent time an ICE session is used, the initial parameters are seeded by the last saved best set of parameters (as illustrated by the dashed arrow). The best set of parameters can also be saved and updated over time, and then provided to and used by other ICE systems, as a starting point for their processing or as the parameters to use for similarly configured systems doing similar ICE sessions.

### Example Embodiments

In one aspect, an intracardiac echocardiography (ICE) catheter system includes a control system disposed in a portable housing, the control system including a battery; an ICE ultrasound circuit connected to the battery, the ICE ultrasound circuit configured to be connected to an ICE catheter and process data received from an ultrasonic array positioned of the ICE catheter; a computing module having electronics and software loaded thereon capable of processing data; and a wireless module connected to the battery and the computing module, the wireless module configured to wirelessly communicate signals to a control counsel in one or more displays that are configured for use in an ICE catheter procedure. In some aspects, the ICE catheter system further includes a durable catheter handle, and a link cable, the link cable having a distal end configured to be connected to the catheter handle and the proximal end configured to be connected to the control system, the link cable configured to communicate signals between the catheter handle and the control system and communicate signals between the catheter connected to the catheter handle in the control system. The control system is configured to operate the catheter handle and the ICE catheter using power from the battery without another energy source. The computing module is configured to determine a processing capability of the control system and determine a processing capability of the control console; and determine operations related to processing ICE images to be performed in the control system and operations related to processing ICE images to be performed in the control console based on the determined processing capabilities of the control system and the control counsel. The computing module is further configured to communicate, via the wireless module, information for processing ICE images in the control console, to the control console.

In one aspect, an intracardiac echocardiography (ICE) catheter system, includes a catheter handle including a control system disposed in the catheter handle, the control system including a battery; an ICE ultrasound circuit connected to the battery, the ICE ultrasound circuit configured to be connected to an ICE catheter and process data received from an ultrasonic array positioned of the ICE catheter; a computing module having electronics and software loaded thereon capable of processing data; and a wireless module connected to the battery and the computing module, the wireless module configured to wirelessly communicate signals to a control counsel in one or more displays that are configured for use in an ICE catheter procedure. The catheter handle is configured to a reusable handle. The control system is configured to operate the catheter handle and the ICE catheter using power from the battery without another energy source. The computing module is configured to determine a processing capability of the control system and determine a processing capability of a control console; and determine operations related to processing ICE images to be performed in the control system and operations related to processing ICE images to be performed in the control console based on the determined processing capabilities of the control system and the control counsel. The computing module is further configured to communicate, via the wireless module, information for processing ICE images to the control console.

In one aspect, a method of processing intracardiac echocardiography (ICE) catheter data includes: determining a processing capability of a plurality of units being used in an ICE session, each of the units having at least one computer hardware processor configured to process ICE data, where a first one of the units receives ICE data during an ICE procedure and the first one of the units is in communication with the other of the plurality of units; providing ICE data from the first one of the units to at least one other of the plurality of units; processing the ICE data on the first one of the plurality of units and the at least one other of the plurality of units; and aggregating processed ICE data from each of the first one of the plurality of units and the at least one other of the plurality of units; and displaying the aggregated processed ICE data as an image on a display. The processing of the ICE data on the first one of the plurality of units and the at least one other of the plurality of units occurs simultaneously. One of the plurality of units is an ICE unit. One of the plurality of units is an ICE catheter handle unit. One of the plurality of units is a console unit. One of the plurality of units is a remote unit. Providing ICE data from the first one of the units to at least one other of the plurality of units comprises providing ICE data from the first one of the units to at least one other of the plurality of units via a wireless communication channel.

### Implementation Considerations

The foregoing description details certain embodiments of the systems, devices, and methods disclosed herein. It will be appreciated, however, that no matter how detailed the foregoing appears in text, the systems, devices, and methods can be practiced in many ways. As is also stated above, it should be noted that the use of particular terminology when describing certain features or aspects of the invention should not be taken to imply that the terminology is being re-defined herein to be restricted to including any specific characteristics of the features or aspects of the technology with which that terminology is associated.

Conditional language such as, among others, “can,” “could,” “might” or “may,” unless specifically stated otherwise, are otherwise understood within the context as used in general to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Headings are included herein for reference and to aid in locating various sections. These headings are not intended to limit the scope of the concepts described with respect thereto. Such concepts may have applicability throughout the entire specification.

Many variations and modifications may be made to the above-described embodiments, the elements of which are to be understood as being among other acceptable examples. All such modifications and variations are intended to be included herein within the scope of this disclosure. The foregoing description details certain embodiments. It will be appreciated, however, that no matter how detailed the foregoing appears in text, the systems and methods can be practiced in many ways. As is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

It will also be understood that, when a feature or element (for example, a structural feature or element) is referred to as being “connected”, “attached” or “coupled” to another feature or element, it may be directly connected, attached or coupled to the other feature or element or intervening features or elements may be present. In contrast, when a feature or element is referred to as being “directly connected”, “directly attached” or “directly coupled” to another feature or element, there may be no intervening features or elements present. Although described or shown with respect to one embodiment, the features and elements so described or shown may apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments and implementations only and is not intended to be limiting. For example, as used herein, the singular forms “a”, “an” and “the” may be intended to include the plural forms as well, unless the context clearly indicates otherwise. It will be further understood that the terms “comprises” and/or “comprising,” when used in this specification, specify the presence of stated features, steps, operations, processes, functions, elements, and/or components, but do not preclude the presence or addition of one or more other features, steps, operations, processes, functions, elements, components, and/or groups thereof. As used herein, the term “and/or” includes any and all combinations of one or more of the associated listed items and may be abbreviated as “/”.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C,” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

Spatially relative terms, such as “forward,” “rearward,” “under,” “below,” “lower,” “over,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features due to the inverted state. Thus, the term “under” may encompass both an orientation of over and under, depending on the point of reference or orientation. The device may be otherwise oriented (rotated 90 degrees or at other orientations) and the spatially relative descriptors used herein interpreted accordingly. Similarly, the terms “upwardly”, “downwardly”, “vertical”, “horizontal” and the like may be used herein for the purpose of explanation only unless specifically indicated otherwise.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise.

For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, may represent endpoints or starting points, and ranges for any combination of the data points. For example, if a particular data point “10” and a particular data point “15” may be disclosed, it is understood that greater than, greater than or equal to, less than, less than or equal to, and equal to 10 and 15 may be considered disclosed as well as between 10 and 15. It is also understood that each unit between two particular units may be also disclosed. For example, if 10 and 15 may be disclosed, then 11, 12, 13, and 14 may be also disclosed.

Although various illustrative embodiments have been disclosed, any of a number of changes may be made to various embodiments without departing from the teachings herein. For example, the order in which various described method steps are performed may be changed or reconfigured in different or alternative embodiments, and in other embodiments one or more method steps may be skipped altogether. Optional or desirable features of various device and system embodiments may be included in some embodiments and not in others. Therefore, the foregoing description is provided primarily for the purpose of example and should not be interpreted to limit the scope of the claims and specific embodiments or particular details or features disclosed.

The examples and illustrations included herein show, by way of illustration and not of limitation, specific embodiments in which the disclosed subject matter may be practiced. As mentioned, other embodiments may be utilized and derived therefrom, such that structural and logical substitutions and changes may be made without departing from the scope of this disclosure. Such embodiments of the disclosed subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve an intended, practical or disclosed purpose, whether explicitly stated or implied, may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The disclosed subject matter has been provided here with reference to one or more features or embodiments. Those skilled in the art will recognize and appreciate that, despite of the detailed nature of the example embodiments provided here, changes and modifications may be applied to said embodiments without limiting or departing from the generally intended scope. These and various other adaptations and combinations of the embodiments provided here are within the scope of the disclosed subject matter as defined by the disclosed elements and features and their full set of equivalents.

1-24. (canceled)

25. An intracardiac echocardiography (ICE) catheter system, comprising:
a control system disposed in a portable housing, the control system including
a battery coupled to components in the portable housing to provide power;
an ICE ultrasound circuit configured to control an ultrasonic array of an ICE catheter in communication with the ICE ultrasound circuit, and to receive and process data signals received from the ultrasonic array;
a computing module comprising one or more hardware processors configured with executable instructions to generate image data from the processed data signals received from the ICE ultrasound circuit;
a wireless module comprising one or more hardware processors configured with executable instructions configured to receive the image data from the computing module and wirelessly communicate signals to a control counsel and to the one or more displays for displaying the image data; and
a catheter handle configured to communicate with the control system, the catheter handle further configured to perform processing operations related to processing ICE images.

26. The ICE catheter system of claim 25, wherein the catheter handle comprises a durable catheter handle having controls thereon and a link cable, the link cable having a distal end configured to be mechanically and electrically connected to the catheter handle and a proximal end configured to be mechanically and electrically connected to the control system, the link cable providing electrical connections to communicate control signals between the catheter handle and the control system and to communicate data signals received from an ICE catheter coupled to the catheter handle to the control system.

27. The ICE catheter system of claim 25, wherein the control system is configured to operate the catheter handle and an ICE catheter coupled to the catheter handle using power from the battery, via the link cable, without receiving power from another energy source.

28. The ICE catheter system of claim 25, further comprising a control console having one or more processors configured to wirelessly communicate control system to receive ICE data.

29. The ICE catheter system of claim 28, wherein the computing module is further configured to
determine a processing capability of the control system;
determine a processing capability of the control console; and
determine operations related to processing ICE images to be performed in the control system and operations related to processing ICE images to be performed in the control console based on the determined processing capabilities of the control system and the control counsel.

30. The ICE catheter system of claim 29, wherein the computing module is further configured to communicate, via the wireless module, information for processing ICE images to the control console.

31. The ICE catheter system of claim 28, wherein the control console further comprises one or more displays for displaying image data received wirelessly from the control system.

32. The ICE catheter system of claim 28, wherein the control console is further configured to communicate controls to an ICE catheter coupled to the catheter handle via the control system and the catheter handle.

33. The ICE catheter system of claim 28, wherein the computing module is further configured to determine a processing capability of the catheter handle, and determine operations related to processing ICE images to be performed in the catheter handle based on the determined processing capabilities of the control system, the control counsel, and the catheter handle.

34. The ICE catheter system of claim 33, wherein the computing module is further configured to communicate information for processing ICE images to the catheter handle.

35. The ICE catheter system of claim 28, further comprising a remote unit having a processing capability, and wherein the computing module is further configured to determine a processing capability of the remote unit and determine operations related to processing ICE images to be performed in the remote unit based on the determined processing capabilities of the control system, the control counsel, the catheter handle, and the remote unit.

36. The ICE catheter system of claim 35, wherein the computing module is further configured to communicate, via the wireless module, information for processing ICE images to the remote unit.

37. A method of processing intracardiac echocardiography (ICE) catheter data, the method comprising:
receiving, during an ICE procedure, ICE data from an ICE catheter at a first unit of a plurality of units, each of the plurality of units having at least one computer hardware processor configured to process ICE data, and the first one of the units is in communication with the other of the plurality of units;
providing ICE data from the first one of the units to at least one other of the plurality of units;
processing the ICE data on the first one of the plurality of units and the at least one other of the plurality of units; and
aggregating processed ICE data from each of the first one of the plurality of units and the at least one other of the plurality of units; and
displaying the aggregated processed ICE data as an image on a display.

38. The method of claim 37, wherein one of the plurality of units is an ICE catheter handle.

39. The method claim 37, further comprising determining a processing capability of each of the plurality of units, wherein providing ICE data from the first one of the units to at least one other of the plurality of units is based on the determined processing capability of each of the plurality of units.

40. The method of claim 37, wherein processing of the ICE data on the first one of the plurality of units and the at least one other of the plurality of units occurs simultaneously.

41. The method of claim 37, wherein one of the plurality of units is a control system disposed in a portable housing.

42. The method of claim 37, further comprising sending commands from the control console to perform functional actions by an ICE catheter coupled to the catheter handle.

43. The method of claim 37, wherein the first unit is a control system, the method further comprising sending commands from a remote control unit to the control system to control one or more functions or processes during an ICE session.

44. The method of claim 43, wherein providing ICE data from the first one of the units to at least one other of the plurality of units comprises providing ICE data from the first one of the units to at least one other of the plurality of units via a wireless communication channel.
